(19) United States
(12) Patent Application Publication
(10) Pub. No.: US 2021/0072244 A1
(43) Pub. Date: Mar. 11, 2021

(54) METHODS AND COMPOSITIONS FOR TREATING MELANOMA RESISTANT

(71) Applicants: INSERM (INSTITUT NATIONAL DE LA SANTÉ ET DE LA RECHERCHE MÉDICALE), Paris (FR); UNIVERSITÉ COTE D'AZUR, NICE (FR)

(72) Inventors: Corine BERTOLOTTO, Nice Cedex 3 (FR); Mickaël OHANNA, Nice Cedex 3 (FR); Robert BALLOTTI, Nice Cedex 3 (FR)

(21) Appl. No.: 16/959,541

(22) PCT Filed: Jan. 3, 2019
(86) PCT No.: PCT/EP2019/050118

§ 371 Date: Jul. 1, 2020

(30) Foreign Application Priority Data
Jan. 4, 2018 (EP) 18305002.0

(51) Int. Cl. G01N 33/574; A61K 31/4545; A61K 31/4409; A61K 31/713

(52) U.S. Cl. CPC G01N 33/5743; A61K 31/4545; G01N 2500/04; A61K 31/713; G01N 2333/91142; A61K 31/4409

(57) ABSTRACT

The present invention relates to a method for treating a subject suffering from melanoma resistant by administering to said subject an inhibitor of NAMPT. Using a global metabolic profiling, inventors have showed that in addition to glycolysis, the BRAF inhibitor, PLX4032, promoted a complex metabolic rewiring of melanoma cells, including protein catabolism and fatty acid synthesis. Importantly, they observed that PLX4032 reduced the levels of nicotinamide adenine dinucleotide (NAD+), an important redox co-factor in numerous metabolic processes, including glycolysis, tricarboxylic acid cycle (TCA) cycle, glutamate metabolism and fatty acid betaoxidation. Pharmacological or genetic inhibition of NAMPT impaired melanoma cell growth, whereas the overexpression of NAMPT dampened the antiproliferative effect of PLX4032. In vivo, the inhibition of NAMPT also prevented the xenograft development of PLX4032-sensitive and -resistant melanoma cells, identifying NAMPT as a potential target for BRAFi-resistant melanomas.

### FIELD OF THE INVENTION

The invention is in the field of cancer. More particularly, the invention relates to methods and compositions for treating melanoma resistant.

### BACKGROUND OF THE INVENTION

Reprogramming of cellular energy metabolism has recently been rediscovered as an emerging hallmark of cancer (Ward and Thompson 2012). In 1930, Otto Warburg identified alterations of energy metabolism in cancer cells (Warburg 1956). For ATP production, cancer cells switch from oxidative phosphorylation (OxPhos) to glycolysis, regardless of the oxygen supply, leading to a process called “aerobic glycolysis”. This switch is frequently associated with an enhanced glucose uptake that compensates for the poor energetic efficiency of glycolysis compared with oxidative phosphorylation. BRAF oncogenic mutations, which increase glycolytic activity in a variety of cancer cells, particularly in melanoma, play a key role in this metabolic switch (Parmenter et al. 2014). The inhibition of glycolysis upon BRAF inhibitor treatment participates in the therapeutic response (Baudy et al. 2012; Parmenter et al. 2014). However, as phenotypically and functionally plastic cells, melanoma cells can rewire their metabolism toward oxidative phosphorylation or glutaminolysis upon BRAF inhibitor exposure, a process that dampens the efficacy of the drug and support tumor growth (Haq et al. 2013; Baenke et al. 2016). Therefore, these alternative metabolic pathways for energy supply, or the restoration of glycolysis (Parmenter et al. 2014) might also contribute to the acquisition of resistance to BRAF inhibitor (BRAFi), leading to treatment failure in patients with BRAF-mutated melanoma. Further, glycolysis, oxidative phosphorylation and glutaminolysis are intertwined with other key metabolic pathways, such as protein catabolism, which can fuel the Krebs cycle and fatty acid synthesis/beta-oxidation pathways (through citrate and Acetyl CoA) that also represent alternative sources of energy. Obtaining a complete view of the different metabolic pathways deregulated upon BRAF inhibition in melanoma cells is pivotal to a better understanding of the mechanisms involved in the implementation of resistances and improvements in antimelanoma therapies.

### SUMMARY OF THE INVENTION

The invention relates to a method for predicting the risk of relapse to a treatment in a subject suffering from melanoma comprising the steps of: i) measuring the expression level of NAMPT in a biological sample obtained from said subject; ii) comparing the expression level measured at step i) with its predetermined reference value, and iii) concluding that the subject is at risk of relapse to the treatment when the expression level of NAMPT is higher than its predetermined reference value or concluding that the subject is not at risk of relapse when the expression level of NAMPT is lower than its predetermined reference value. In particular, the invention is defined by claims.

### DETAILED DESCRIPTION OF THE INVENTION

Using a global metabolic profiling, inventors have shown that in addition to glycolysis, the BRAF inhibitor, PLX4032, promoted a complex metabolic rewiring of melanoma cells, including protein catabolism and fatty acid synthesis. These observations reveal the implementation of alternative energy production pathways that might dampen the anti-proliferative effect of BRAFi. Importantly, they observed that PLX4032 reduced the levels of nicotinamide adenine dinucleotide (NAD+), an important redox co-factor in numerous metabolic processes, including glycolysis, tricarboxylic acid cycle (TCA), glutamate metabolism and fatty acid betaoxidation. They also showed that PLX4032 and other ERK pathway inhibitors inhibited the expression of nicotinamide phosphoribosyltransferase (NAMPT), the rate-limiting enzyme in the NAD+ salvage pathway. Inversely, forced expression of BRAFV600E in normal human melanocytes increased NAMPT expression and NAD+ production. Noticeably, pharmacological or genetic inhibition of NAMPT impaired melanoma cell growth, whereas the overexpression of NAMPT dampened the antiproliferative effect of PLX4032. In vivo, the inhibition of NAMPT also prevented the xenograft development of PLX4032-sensitive and -resistant melanoma cells, identifying NAMPT as a potential target for BRAFi-resistant melanomas.

Accordingly, in a first aspect, the invention relates to a method for predicting the risk of relapse to a treatment in a subject suffering from melanoma comprising the steps of: i) measuring the activity and/or expression level of NAMPT in a biological sample obtained from said subject; ii) comparing the expression level measured at step i) with its predetermined reference value, and iii) concluding that the subject is at risk of relapse to the treatment when the expression level of NAMPT is higher than its predetermined reference value or concluding that the subject is not at risk of relapse when the expression level of NAMPT is lower than its predetermined reference value.

In a particular embodiment, the activity of NAMPT is determined by measuring the production level of NAD. Accordingly, the invention relates to a method for predicting the risk of relapse to a treatment in a subject suffering from melanoma comprising the steps of: i) measuring the activity of NAMPT by determining the production level of NAD+ in a biological sample obtained from said subject; ii) comparing the production level of NAD+ measured at step i) with its predetermined reference value, and iii) concluding that the subject is at risk of relapse to the treatment when the production level of NAD+ is higher than its predetermined reference value or concluding that the subject is not at risk of relapse when the production level of NAD+ is lower than its predetermined reference value. As used herein, the term “predicting” means that the subject to be analyzed by the method of the invention is allocated either into the group of subjects who will relapse, or into a group of subjects who will not relapse after a treatment.

As used herein, the term “risk” in the context of the present invention, relates to the probability that an event will occur over a specific time period, as in the conversion to relapse, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed. Odds ratios, the proportion of positive events to negative events for a given test result, are also commonly used (odds are according to the formula p/(l−p) where p is the probability of event and (l−p) is the probability of no event) to no-conversion. “Risk evaluation,” or “evaluation of risk” in the context of the present invention encompasses making a prediction of the probability, odds, or likelihood that an event or disease state may occur, the rate of occurrence of the event or conversion from one disease state to another, i.e., from a normal condition to relapse or to one at risk of developing relapse. Risk evaluation can also comprise prediction of future clinical parameters, traditional laboratory risk factor values, or other indices of relapse, either in absolute or relative terms in reference to a previously measured population. The methods of the present invention may be used to make continuous or categorical measurements of the risk of conversion to relapse, thus diagnosing and defining the risk spectrum of a category of subjects defined as being at risk of having relapse. In the categorical scenario, the invention can be used to discriminate between normal and other subject cohorts at higher risk of having relapse. In some embodiments, the present invention may be used so as to discriminate those at risk of having relapse from normal, or those having relapse disease from normal.

As used herein, the term “melanoma” also known as malignant melanoma, refers to a type of cancer that develops from the pigment-containing cells, called melanocytes. There are three general categories of melanoma: 1) cutaneous melanoma which corresponds to melanoma of the skin; it is the most common type of melanoma; 2) mucosal melanoma which can occur in any mucous membrane of the body, including the nasal passages, the throat, the vagina, the anus, or in the mouth; and 3) ocular melanoma also known as uveal melanoma or choroidal melanoma and conjunctival, is a rare form of melanoma that occurs in the eye. In a particular embodiment, the melanoma is cutaneous melanoma.

As used herein, the term “NAMPT” also known as pre-B-cell colony-enhancing factor 1 (PBEF1) or visfatin refers to nicotinamide phosphoribosyltransferase. It is an enzyme that in humans is encoded by the PBEF1 gene. The protein NAMPT catalyzes the condensation of nicotinamide with 5-phosphoribosyl-1-pyrophosphate to yield nicotinamide mononucleotide, one step in the biosynthesis of nicotinamide adenine dinucleotide. The protein belongs to the nicotinic acid phosphoribosyltransferase (NAPRTase) family and is thought to be involved in many important biological processes, including metabolism, stress response and aging. NAMPT has a pivotal role in the recycling pathway allowing NAD generation from nicotinamide. NAMPT occupies a central position in controlling the activity of several NAD-dependent enzymes (Galli et al., 2010). Accordingly, the activity of NAMPT can be determined by measuring the production level of NAD. The naturally occurring human NAMPT gene has a nucleotide sequence as shown in Genbank Accession number NM_005746.2 and the naturally occurring human NAMPT protein has an amino acid sequence as shown in Genbank Accession number NP_005737.1. The murine nucleotide and amino acid sequences have also been described (Genbank Accession numbers NM_021524.2 and NP_067499.2).

As used herein, the term “NAD” refers to nicotinamide adenine dinucleotide. It is a coenzyme involved in redox reactions. NAD+ is synthesized through two metabolic pathways: de novo pathway from amino acids or in salvage pathways by recycling preformed components such as nicotinamide back to NAD+ (Canto. C et al 2015).

As used herein, the term “subject” denotes a mammal, such as a rodent, a feline, a canine, and a primate. Particularly, the subject according to the invention is a human. More particularly, the subject according to the invention has or susceptible to have melanoma. In a particular embodiment, the subject has or susceptible to have melanoma resistant to the treatments. As used herein, the term “resistant melanoma” refers to melanoma which does not respond to a treatment. The cancer may be resistant at the beginning of treatment or it may become resistant during treatment. The resistance to drug leads to rapid progression of metastatic of melanoma. The resistance of cancer for the medication is caused by acquisition of novel mutations or change in gene expression which are involved in the proliferation, division, migration or differentiation of cells. In the context of the invention, the resistance of melanoma is caused by the mutations (single or double) in the following genes: BRAF, MEK or NRAS. The subject having a melanoma resistant is identified by standard criteria. The standard criteria for resistance, for example, are Response Evaluation Criteria In Solid Tumors (RECIST) criteria, published by an international consortium including NCI.

As used herein, the term “relapse” refers to the return of signs and symptoms of a disease after a subject has enjoyed a remission after a treatment. Thus, if initially the target disease is alleviated or healed, or progression of the disease was halted or slowed down, and subsequently the disease or one or more characteristics of the disease return, the subject is referred to as being “relapsed.” This kind of relapse is also called resistant. In some embodiments, the method of the present invention is particularly suitable for predicting the risk of relapse when the subject was or is treated with at least one agent selected from the group consisting of: PLX4032, immunotherapy and combined treatment.

As used herein, the term “PLX4032” also known as vemurafenib, RG7204 or RO5185426 refers to a small molecule which inhibits B-Raf kinase. PLX4032 has the FDA approval on 2011 for the treatment of late-stage melanoma and is commercialized under the trade name Zelboraf. This molecule has the CAS number 918504-65-1 and the following chemical formula in the art C₂₃H₁₈C₁F₂N₃O₃S.

As used herein, the term “immunotherapy” refers to the use of the compounds which modulate the immune system. Typically, the following types of immunotherapy can be used to treat melanoma: monoclonal antibodies, immune checkpoint inhibitors or cancer vaccines. In a particular embodiment, the method according to the invention is suitable to predict the risk of relapse to the treatment with immune checkpoint inhibitors. As used herein, the term “immune checkpoint inhibitor” refers to molecules that totally or partially reduce, inhibit, interfere with or modulate one or more immune checkpoint proteins. As used herein, the term “immune checkpoint protein” has its general meaning in the art and refers to a molecule that is expressed by T cells in that either turn up a signal (stimulatory checkpoint molecules) or turn down a signal (inhibitory checkpoint molecules). Immune checkpoint molecules are recognized in the art to constitute immune checkpoint pathways similar to the CTLA-4 and PD-1 dependent pathways (see e.g. Pardoll, 2012. Nature Rev Cancer 12:252-264; Mellman et al., 2011. Nature 480:480-489). Examples of stimulatory checkpoint include CD27 CD28 CD40, CD122, CD137, OX40, GITR, and ICOS. Examples of inhibitory checkpoint molecules include A2AR, B7-H3, B7-H4, BTLA, CTLA-4, CD277, IDO, KIR, PD-1, LAG-3, TIM-3 and VISTA. The Adenosine A2A receptor (A2AR) is regarded as an important checkpoint in cancer therapy because adenosine in the immune microenvironment, leading to the activation of the A2a receptor, is negative immune feedback loop and the tumor microenvironment has relatively high concentrations of adenosine. B7-H3, also called CD276, was originally understood to be a co-stimulatory molecule but is now regarded as co-inhibitory. B7-H4, also called VTCN1, is expressed by tumor cells and tumor-associated macrophages and plays a role in tumour escape. B and T Lymphocyte Attenuator (BTLA) and also called CD272, has HVEM (Herpesvirus Entry Mediator) as its ligand. Surface expression of BTLA is gradually downregulated during differentiation of human CD8+ T cells from the naive to effector cell phenotype, however tumor-specific human CD8+ T cells express high levels of BTLA. CTLA-4, Cytotoxic T-Lymphocyte-Associated protein 4 and also called CD152. Expression of CTLA-4 on Treg cells serves to control T cell proliferation. IDO, Indoleamine 2,3-dioxygenase, is a tryptophan catabolic enzyme. A related immune-inhibitory enzymes. Another important molecule is TDO, tryptophan 2,3-dioxygenase. IDO is known to suppress T and NK cells, generate and activate Tregs and myeloid-derived suppressor cells, and promote tumour angiogenesis. KIR, Killer-cell Immunoglobulin-like Receptor, is a receptor for MHC Class I molecules on Natural Killer cells. LAG3, Lymphocyte Activation Gene-3, works to suppress an immune response by action to Tregs as well as direct effects on CD8+ T cells. PD-1, Programmed Death 1 (PD-1) receptor, has two ligands, PD-L1 and PD-L2. This checkpoint is the target of Merck & Co.'s melanoma drug Keytruda, which gained FDA approval in September 2014. An advantage of targeting PD-1 is that it can restore immune function in the tumor microenvironment. TIM-3, short for T-cell Immunoglobulin domain and Mucin domain 3, expresses on activated human CD4+ T cells and regulates Th1 and Th17 cytokines. TIM-3 acts as a negative regulator of Th1/Tc1 function by triggering cell death upon interaction with its ligand, galectin-9. VISTA, Short for V-domain Ig suppressor of T cell activation, VISTA is primarily expressed on hematopoietic cells so that consistent expression of VISTA on leukocytes within tumors may allow VISTA blockade to be effective across a broad range of solid tumors. Tumor cells often take advantage of these checkpoints to escape detection by the immune system. Thus, inhibiting a checkpoint protein on the immune system may enhance the anti-tumor T-cell response.

In some embodiments, an immune checkpoint inhibitor refers to any compound inhibiting the function of an immune checkpoint protein Inhibition includes reduction of function and full blockade. In some embodiments, the immune checkpoint inhibitor could be an antibody, synthetic or native sequence peptides, small molecules or aptamers which bind to the immune checkpoint proteins and their ligands.

In a particular embodiment, the immune checkpoint inhibitor is an antibody.

Typically, antibodies are directed against A2AR, B7-H3, B7-H4, BTLA, CTLA-4, CD277, IDO, KIR, PD-1, LAG-3, TIM-3 or VISTA.

In a particular embodiment, the immune checkpoint inhibitor is an anti-PD-1 antibody such as described in WO2011082400, WO2006121168, WO2015035606, WO2004056875, WO2010036959, WO2009114335, WO2010089411, WO2008156712, WO2011110621, WO2014055648 and WO2014194302. Examples of anti-PD-1 antibodies which are commercialized: Nivolumab (Opdivo®, BMS), Pembrolizumab (also called Lambrolizumab, KEYTRUDA® or MK-3475, MERCK).

In some embodiments, the immune checkpoint inhibitor is an anti-PD-L1 antibody such as described in WO2013079174, WO2010077634, WO2004004771, WO2014195852, WO2010036959, WO2011066389, WO2007005874, WO2015048520, U.S. Pat. No. 8,617,546 and

WO2014055897. Examples of anti-PD-L1 antibodies which are on clinical trial: Atezolizumab (MPDL3280A, Genentech/Roche), Durvalumab (AZD9291, AstraZeneca), Avelumab (also known as MSB0010718C, Merck) and BMS-936559 (BMS).

In some embodiments, the immune checkpoint inhibitor is an anti-PD-L2 antibody such as described in U.S. Pat. Nos. 7,709,214, 7,432,059 and 8,552,154.

In the context of the invention, the immune checkpoint inhibitor inhibits Tim-3 or its ligand.

In a particular embodiment, the immune checkpoint inhibitor is an anti-Tim-3 antibody such as described in WO03063792, WO2011155607, WO2015117002, WO2010117057 and WO2013006490.

In some embodiments, the immune checkpoint inhibitor is a small organic molecule.

The term “small organic molecule” as used herein, refers to a molecule of a size comparable to those organic molecules generally used in pharmaceuticals. The term excludes biological macro molecules (e. g. proteins, nucleic acids, etc.). Typically, small organic molecules range in size up to about 5000 Da, more preferably up to 2000 Da, and most preferably up to about 1000 Da.

Typically, the small organic molecules interfere with transduction pathway of A2AR, B7-H3, B7-H4, BTLA, CTLA-4, CD277, IDO, KIR, PD-1, LAG-3, TIM-3 or VISTA.

In a particular embodiment, small organic molecules interfere with transduction pathway of PD-1 and Tim-3. For example, they can interfere with molecules, receptors or enzymes involved in PD-1 and Tim-3 pathway.

In a particular embodiment, the small organic molecules interfere with Indoleamine-pyrrole 2,3-dioxygenase (IDO) inhibitor. IDO is involved in the tryptophan catabolism (Liu et al 2010, Vacchelli et al 2014, Zhai et al 2015). Examples of IDO inhibitors are described in WO 2014150677. Examples of IDO inhibitors include without limitation 1-methyl-tryptophan (IMT), β-(3-benzofuranyl)-alanine, P-(3-benzo(b)thienyl)-alanine), 6-nitro-tryptophan, 6-fluoro-tryptophan, 4-methyl-tryptophan, 5-methyl tryptophan, 6-methyl-tryptophan, 5-methoxy-tryptophan, 5-hydroxy-tryptophan, indole 3-carbinol, 3,3′-diindolylmethane, epigallocatechin gallate, 5-Br-4-Cl-indoxyl 1,3-diacetate, 9-vinylcarbazole, acemetacin, 5-bromo-tryptophan, 5-bromoindoxyl diacetate, 3-Amino-naphtoic acid, pyrrolidine dithiocarbamate, 4-phenylimidazole a brassinin derivative, a thiohydantoin derivative, a β-carboline derivative or a brassilexin derivative. In a particular embodiment, the IDO inhibitor is selected from 1-methyl-tryptophan, β-(3-benzofuranyl)-alanine, 6-nitro-L-tryptophan, 3-Amino-naphtoic acid and β-[3-benzo(b)thienyl]-alanine or a derivative or prodrug thereof.

In a particular embodiment, the inhibitor of IDO is Epacadostat, (INCB24360, INCB024360) has the following chemical formula in the art and refers to —N-(3-bromo-4-fluorophenyl)-N′-hydroxy-4-{[2-(sulfamoylamino)-éthyl]amino}-1,2,5-oxadiazole-3 carboximidamide:

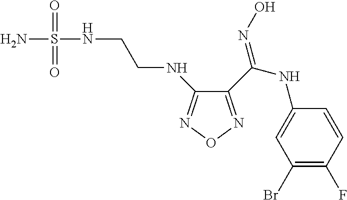 [SMILES: NS(=O)(=O)NCCNc1nonc1/C(=N/O)Nc1ccc(F)c(Br)c1]

In a particular embodiment, the inhibitor is BGB324, also called R428, such as described in WO2009054864, refers to 1H-1,2,4-Triazole-3,5-diamine, 1-(6,7-dihydro-5H-benzo[6,7]cyclohepta[1,2-c]pyridazin-3-yl)-N3-[(7S)-6,7,8,9-tetrahydro-7-(1-pyrrolidinyl)-5H-benzocyclohepten-2-yl]- and has the following formula in the art:

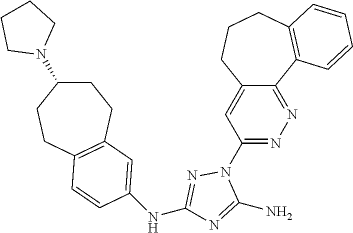 [SMILES: Nc1nc(Nc2ccc3c(c2)CC[C@@H](N2CCCC2)CC3)nn1-c1cc2c(nn1)-c1ccccc1CCC2]

In a particular embodiment, the inhibitor is CA-170 (or AUPM-170): an oral, small molecule immune checkpoint antagonist targeting programmed death ligand-1 (PD-L1) and V-domain Ig suppressor of T cell activation (VISTA) (Liu et al 2015). Preclinical data of CA-170 are presented by Curis Collaborator and Aurigene on November at ACR-NCI-EORTC International Conference on Molecular Targets and Cancer Therapeutics.

In some embodiments, the immune checkpoint inhibitor is an aptamer.

Typically, the aptamers are directed against A2AR, B7-H3, B7-H4, BTLA, CTLA-4, CD277, IDO, KIR, PD-1, LAG-3, TIM-3 or VISTA.

In a particular embodiment, aptamers are DNA aptamers such as described in Prodeus et al 2015. A major disadvantage of aptamers as therapeutic entities is their poor pharmacokinetic profiles, as these short DNA strands are rapidly removed from circulation due to renal filtration. Thus, aptamers according to the invention are conjugated to with high molecular weight polymers such as polyethylene glycol (PEG). In a particular embodiment, the aptamer is an anti-PD-1 aptamer. Particularly, the anti-PD-1 aptamer is MP7 pegylated as described in Prodeus et al 2015.

As used herein, the terms “combined treatment”, “combined therapy” or “therapy combination” refer to a treatment that uses more than one medication. The combined therapy may be dual therapy or bi-therapy.

The medications used in the combined treatment according to the invention are administered to the subject simultaneously, separately or sequentially.

As used herein, the term “administration simultaneously” refers to administration of 2 active ingredients by the same route and at the same time or at substantially the same time. The term “administration separately” refers to an administration of 2 active ingredients at the same time or at substantially the same time by different routes. The term “administration sequentially” refers to an administration of 2 active ingredients at different times, the administration route being identical or different.

In the context of the invention, the melanoma is resistant to a combined treatment characterized by using an inhibitor of BRAF mutation and an inhibitor of MEK. For example, the combined treatment may be a combination of vemurafenib and cotellic. BRAF is a member of the Raf kinase family of serine/threonine-specific protein kinases. This protein plays a role in regulating the MAP kinase/ERKs signaling pathway, which affects cell division, differentiation, and secretion. A number of mutations in BRAF are known. In particular, the V600E mutation is prominent. Other mutations which have been found are R461I, I462S, G463E, G463V, G465A, G465E, G465V, G468A, G468E, N580S, E585K, D593V, F594L, G595R, L596V, T598I, V599D, V599E, V599K, V599R, K600E, A727V, and most of these mutations are clustered to two regions: the glycine-rich P loop of the N lobe and the activation segment and flanking regions. In a particular embodiment, the BRAF mutation is V600E. The inhibitors of BRAF mutations are well known in the art. In a particular embodiment, the melanoma is resistant to a treatment with vemurafenib. Vemurafenib also known as PLX4032, RG7204 or RO5185426 and commercialized by Roche as Zelboraf. In a particular embodiment, the melanoma is resistant to a treatment with dacarbazine. Dacarbazine also known as imidazole carboxamide is commercialized as DTIC-Dome by Bayer. In a particular embodiment, the melanoma is resistant to a treatment with dabrafenib also known as tafinlar which is commercialized by Novartis.

MEK refers to Mitogen-activated protein kinase kinase, also known as MAP2K, MEK, MAPKK. It is a kinase enzyme which phosphorylates mitogen-activated protein kinase (MAPK). MEK is activated in melanoma. The inhibitors of MEK are well known in the art. In a particular embodiment, the melanoma is resistant to a treatment with trametinib also known as mekinist and GSK1120212 which is commercialized by GSK. In a particular embodiment, the melanoma is resistant to a treatment with cobimetinib also known as cotellic commercialized by Genentech. In a particular embodiment, the melanoma is resistant to a treatment with Binimetinib also knowns as MEK162, ARRY-162 is developed by Array Biopharma.

In a particular embodiment, the treatment refers to the use of inhibitors of NRAS. The NRAS gene is in the Ras family of oncogene and involved in regulating cell division. NRAS mutations in codons 12, 13, and 61 arise in 15-20% of all melanomas. The inhibitors of BRAF mutation or MEK are used to treat the melanoma with NRAS mutations.

As used herein, the term “expression level” refers to the expression level of NAMPT. Typically, the expression level of the NAMPT gene may be determined by any technology known by a person skilled in the art. In particular, each gene expression level may be measured at the genomic and/or nucleic and/or protein level. In a particular embodiment, the expression level of gene is determined by measuring the amount of nucleic acid transcripts of each gene. In another embodiment, the expression level is determined by measuring the amount of each gene corresponding protein. The amount of nucleic acid transcripts can be measured by any technology known by a man skilled in the art. In particular, the measure may be carried out directly on an extracted messenger RNA (mRNA) sample, or on retrotranscribed complementary DNA (cDNA) prepared from extracted mRNA by technologies well-known in the art. From the mRNA or cDNA sample, the amount of nucleic acid transcripts may be measured using any technology known by a man skilled in the art, including nucleic microarrays, quantitative PCR, microfluidic cards, and hybridization with a labelled probe. In a particular embodiment, the expression level is determined by using quantitative PCR. Quantitative, or real-time, PCR is a well-known and easily available technology for those skilled in the art and does not need a precise description. Methods for determining the quantity of mRNA are well known in the art. For example the nucleic acid contained in the biological sample is first extracted according to standard methods, for example using lytic enzymes or chemical solutions or extracted by nucleic-acid-binding resins following the manufacturer's instructions. The extracted mRNA is then detected by hybridization (e. g., Northern blot analysis) and/or amplification (e.g., RT-PCR). Preferably quantitative or semi-quantitative RT-PCR is preferred. Real-time quantitative or semi-quantitative RT-PCR is particularly advantageous. Other methods of amplification include ligase chain reaction (LCR), transcription-mediated amplification (TMA), strand displacement amplification (SDA) and nucleic acid sequence based amplification (NASBA). Nucleic acids having at least 10 nucleotides and exhibiting sequence complementarity or homology to the mRNA of interest herein find utility as hybridization probes or amplification primers. It is understood that such nucleic acids do not need to be identical, but are typically at least about 80% identical to the homologous region of comparable size, more preferably 85% identical and even more preferably 90-95% identical. In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin). Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate). The nucleic acid primers or probes used in the above amplification and detection method may be assembled as a kit. Such a kit includes consensus primers and molecular probes. A kit also includes the components necessary to determine if amplification has occurred. The kit may also include, for example, PCR buffers and enzymes; positive control sequences, reaction control primers; and instructions for amplifying and detecting the specific sequences. In a particular embodiment, the method of the invention comprises the steps of providing total RNAs extracted from a biological sample and subjecting the RNAs to amplification and hybridization to specific probes, more particularly by means of a quantitative or semi-quantitative RT-PCR. In another embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g. the review by Hoheisel, Nature Reviews, Genetics, 2006, 7:200-210). The methods for the measure of the production of NAD+ are known in the art. The production of NAD+ can be determined by using kits, such as kit ab65348 (colorimetric kit) or EnzyChrom™ NAD/NADH Assay Kit.

As used herein, the term “biological sample” refers to any sample obtained from a subject, such as a serum sample, a plasma sample, a urine sample, a blood sample, a lymph sample, or a tissue biopsy. In a particular embodiment, biological sample for the determination of an expression level include samples such as a blood sample, a lymph sample, or a biopsy. In a particular embodiment, the biological sample is a blood sample, more particularly, peripheral blood mononuclear cells (PBMC). Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis, which will preferentially lyse red blood cells. Such procedures are known to the experts in the art.

As used herein, the term “the predetermined reference value” refers to a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement of cell densities in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after quantifying the cell density in a group of reference, one can use algorithmic analysis for the statistic treatment of the measured densities in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is quite high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, the predetermined reference value is determined by carrying out a method comprising the steps of

a) providing a collection of biological samples from subject suffering from melanoma;

b) providing, for each biological samples provided at step a), information relating to the actual clinical outcome for the corresponding subject (i.e. the duration of the disease-free survival (DFS) and/or the overall survival (OS));

c) providing a serial of arbitrary quantification values;

d) quantifying the cell density for each biological samples contained in the collection provided at step a);

e) classifying said biological samples in two groups for one specific arbitrary quantification value provided at step c), respectively: (i) a first group comprising biological samples that exhibit a quantification value for level that is lower than the said arbitrary quantification value contained in the said serial of quantification values; (ii) a second group comprising biological samples that exhibit a quantification value for said level that is higher than the said arbitrary quantification value contained in the said serial of quantification values; whereby two groups of biological samples are obtained for the said specific quantification value, wherein the biological samples of each group are separately enumerated;

f) calculating the statistical significance between (i) the quantification value obtained at step e) and (ii) the actual clinical outcome of the subjects from which biological samples contained in the first and second groups defined at step f) derive;

g) reiterating steps f) and g) until every arbitrary quantification value provided at step d) is tested;

h) setting the said predetermined reference value as consisting of the arbitrary quantification value for which the highest statistical significance (most significant P-value obtained with a log-rank test, significance when P<0.05) has been calculated at step g).

For example the cell density has been assessed for 100 biological samples of 100 subjects. The 100 samples are ranked according to the cell density. Sample 1 has the highest density and sample 100 has the lowest density. A first grouping provides two subsets: on one side sample Nr 1 and on the other side the 99 other samples. The next grouping provides on one side samples 1 and 2 and on the other side the 98 remaining samples etc., until the last grouping: on one side samples 1 to 99 and on the other side sample Nr 100. According to the information relating to the actual clinical outcome for the corresponding cancer subject, Kaplan-Meier curves are prepared for each of the 99 groups of two subsets. Also for each of the 99 groups, the p value between both subsets was calculated (log-rank test). The predetermined reference value is then selected such as the discrimination based on the criterion of the minimum P-value is the strongest. In other terms, the cell density corresponding to the boundary between both subsets for which the P-value is minimum is considered as the predetermined reference value. It should be noted that the predetermined reference value is not necessarily the median value of cell densities. Thus in some embodiments, the predetermined reference value thus allows discrimination between a poor and a good prognosis with respect to DFS and OS for a subject. Practically, high statistical significance values (e.g. low P values) are generally obtained for a range of successive arbitrary quantification values, and not only for a single arbitrary quantification value. Thus, in one alternative embodiment of the invention, instead of using a definite predetermined reference value, a range of values is provided. Therefore, a minimal statistical significance value (minimal threshold of significance, e.g. maximal threshold P value) is arbitrarily set and a range of a plurality of arbitrary quantification values for which the statistical significance value calculated at step g) is higher (more significant, e.g. lower P-value) are retained, so that a range of quantification values is provided. This range of quantification values includes a “cut-off” value as described above. For example, according to this specific embodiment of a “cut-off” value, the outcome can be determined by comparing the cell density with the range of values which are identified. In some embodiments, a cut-off value thus consists of a range of quantification values, e.g. centered on the quantification value for which the highest statistical significance value is found (e.g. generally the minimum P-value which is found).

Method for Treating Melanoma Resistant

Inventors have shown that NAMPT inhibition triggers death of PLX4032-sensitive and -resistant melanoma cells, while NAMPT forced expression renders BRAFV600E melanoma cells resistant to BRAF inhibition.

Accordingly, in a second aspect, the invention relates to a method for treating a subject suffering from melanoma resistant comprising a step of administering said subject with a therapeutically effective amount of nicotinamide phosphoribosyl transferase (NAMPT) inhibitor.

In a particular embodiment, the invention relates to a method for treating a subject suffering from melanoma resistant comprising following steps: i) determining whether a subject is at risk of relapse to a treatment according to the method, and ii) administering to the subject identified as having a risk of relapse a therapeutically effective amount of nicotinamide phosphoribosyl transferase (NAMPT) inhibitor.

As used herein, the terms “treating” or “treatment” refer to both prophylactic or preventive treatment as well as curative or disease modifying treatment, including treatment of subject at risk of contracting the disease or suspected to have contracted the disease as well as subject who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a subject during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a subject during treatment of an illness, e.g., to keep the subject in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., pain, disease manifestation, etc.]).

As used herein, the term “subject” denotes a mammal, such as a rodent, a feline, a canine, and a primate. Particularly, the subject according to the invention is a human. More particularly, the subject according to the invention has or susceptible to have melanoma. In a particular embodiment, the subject has or susceptible to have melanoma resistant to the treatments.

As used herein, the term “nicotinamide phosphoribosyl transferase (NAMPT) inhibitor” refers to a natural or synthetic compound that has a biological effect to inhibit the activity or the expression of NAMPT. More particularly, such compound by inhibiting NAMPT activity induces a suppression of NAD+ production in a time dependent manner and sustained reduction of NAD+ levels leads to loss of ATP and ultimately cell death. Accordingly, such inhibition decreases the survival and the proliferation of melanoma cells resistant to the classical treatments. In a particular embodiment, the inhibitor of NAMPT is a peptide, peptidomimetic, small organic molecule, antibody, aptamers, siRNA or antisense oligonucleotide. The term “peptidomimetic” refers to a small protein-like chain designed to mimic a peptide. In a particular embodiment, the inhibitor of NAMPT is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity.

In a particular embodiment, the inhibitor of NAMPT is a small organic molecule. The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da. In a particular embodiment, the small molecule is FK866. As used herein, the term “FK866” also known as APO866 or daporinad is in phase II clinical trials by Onxeo for the treatment of Cutaneous T-cell Lymphoma. The Cas number of this molecule is 658084-64-1. This molecule has the following chemical formula and structure in the art: C₂₄H₂₉N₃O₂:

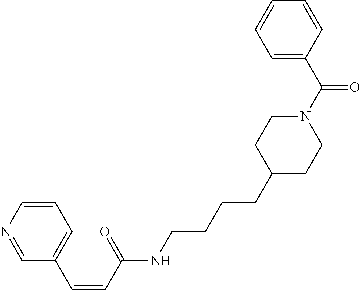 [SMILES: O=C(/C=C\c1cccnc1)NCCCCC1CCN(C(=O)c2ccccc2)CC1]

In another embodiment, the small molecule is CHS 828. As used herein, the term “CHS 828” also known as GMX 1778, has the Cas number 200484-11-3 and the following chemical formula and structure in the art: C19H22ClN5O:

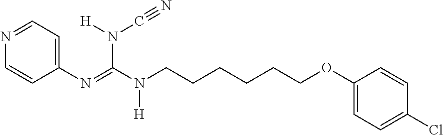 [SMILES: N#CN/C(=N\c1ccncc1)NCCCCCCOc1ccc(Cl)cc1]

In a particular embodiment, the small molecule is KPT-9274. KPT-9274 has the Cas number 1643913-93-2 and the following chemical formula and structure in the art C35H29F3N403:

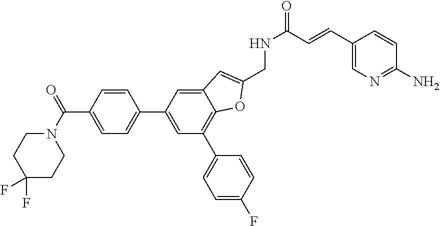 [SMILES: Nc1ccc(/C=C/C(=O)NCc2cc3cc(-c4ccc(C(=O)N5CCC(F)(F)CC5)cc4)cc(-c4ccc(F)cc4)c3o2)cn1]

In some embodiments, the inhibitor of NAMPT expression is a short hairpin RNA (shRNA), a small interfering RNA (siRNA) or an antisense oligonucleotide which inhibits the expression of NAMPT. In a particular embodiment, the inhibitor of NAMPT expression is siRNA. A short hairpin RNA (shRNA) is a sequence of RNA that makes a tight hairpin turn that can be used to silence gene expression via RNA interference. shRNA is generally expressed using a vector introduced into cells, wherein the vector utilizes the U6 promoter to ensure that the shRNA is always expressed. This vector is usually passed on to daughter cells, allowing the gene silencing to be inherited. The shRNA hairpin structure is cleaved by the cellular machinery into siRNA, which is then bound to the RNA-induced silencing complex (RISC). This complex binds to and cleaves mRNAs that match the siRNA to which it is bound. Small interfering RNA (siRNA), sometimes known as short interfering RNA or silencing RNA, are a class of 20-25 nucleotide-long double-stranded RNA molecules that play a variety of roles in biology. Most notably, siRNA is involved in the RNA interference (RNAi) pathway whereby the siRNA interferes with the expression of a specific gene. Anti-sense oligonucleotides include anti-sense RNA molecules and anti-sense DNA molecules, would act to directly block the translation of the targeted mRNA by binding thereto and thus preventing protein translation or increasing mRNA degradation, thus decreasing the level of the targeted protein, and thus activity, in a cell. For example, antisense oligonucleotides of at least about 15 bases and complementary to unique regions of the mRNA transcript sequence can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Antisense oligonucleotides, siRNAs, shRNAs of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically mast cells. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In some embodiments, the inhibitor of NAMPT expression is an endonuclease. In the last few years, staggering advances in sequencing technologies have provided an unprecedentedly detailed overview of the multiple genetic aberrations in cancer. By considerably expanding the list of new potential oncogenes and tumor suppressor genes, these new data strongly emphasize the need of fast and reliable strategies to characterize the normal and pathological function of these genes and assess their role, in particular as driving factors during oncogenesis. As an alternative to more conventional approaches, such as cDNA overexpression or downregulation by RNA interference, the new technologies provide the means to recreate the actual mutations observed in cancer through direct manipulation of the genome. Indeed, natural and engineered nuclease enzymes have attracted considerable attention in the recent years. The mechanism behind endonuclease-based genome inactivating generally requires a first step of DNA single or double strand break, which can then trigger two distinct cellular mechanisms for DNA repair, which can be exploited for DNA inactivating: the error-prone non-homologous end-joining (NHEJ) and the high-fidelity homology-directed repair (HDR).

In a particular embodiment, the endonuclease is CRISPR-cas9. As used herein, the term “CRISPR-cas9” has its general meaning in the art and refers to clustered regularly interspaced short palindromic repeats associated which are the segments of prokaryotic DNA containing short repetitions of base sequences.

In some embodiment, the endonuclease is CRISPR-cas9 which is from Streptococcus pyogenes. The CRISPR/Cas9 system has been described in U.S. Pat. No. 8,697,359 B1 and US 2014/0068797. Originally an adaptive immune system in prokaryotes (Barrangou and Marraffini, 2014), CRISPR has been recently engineered into a new powerful tool for genome editing. It has already been successfully used to target important genes in many cell lines and organisms, including human (Mali et al., 2013, Science, Vol. 339: 823-826), bacteria (Fabre et al., 2014, PLoS Negl. Trop. Dis., Vol. 8:e2671.), zebrafish (Hwang et al., 2013, PLoS One, Vol. 8:e68708.), C. elegans (Hai et al., 2014 Cell Res. doi: 10.1038/cr.2014.11.), bacteria (Fabre et al., 2014, PLoS Negl. Trop. Dis., Vol. 8:e2671.), plants (Mali et al., 2013, Science, Vol. 339: 823-826), Xenopus tropicalis (Guo et al., 2014, Development, Vol. 141: 707-714.), yeast (DiCarlo et al., 2013, Nucleic Acids Res., Vol. 41: 4336-4343.), Drosophila (Gratz et al., 2014 Genetics, doi:10.1534/genetics.113.160713), monkeys (Niu et al., 2014, Cell, Vol. 156: 836-843.), rabbits (Yang et al., 2014, J. Mol. Cell Biol., Vol. 6: 97-99.), pigs (Hai et al., 2014, Cell Res. doi: 10.1038/cr.2014.11.), rats (Ma et al., 2014, Cell Res., Vol. 24: 122-125.) and mice (Mashiko et al., 2014, Dev. Growth Differ. Vol. 56: 122-129.). Several groups have now taken advantage of this method to introduce single point mutations (deletions or insertions) in a particular target gene, via a single gRNA. Using a pair of gRNA-directed Cas9 nucleases instead, it is also possible to induce large deletions or genomic rearrangements, such as inversions or translocations. A recent exciting development is the use of the dCas9 version of the CRISPR/Cas9 system to target protein domains for transcriptional regulation, epigenetic modification, and microscopic visualization of specific genome loci.

In some embodiment, the endonuclease is CRISPR-Cpf1 which is the more recently characterized CRISPR from Provotella and Francisella 1 (Cpf1) in Zetsche et al. (“Cpf1 is a Single RNA-guided Endonuclease of a Class 2 CRISPR-Cas9 System (2015); Cell; 163, 1-13).

In some embodiments, the inhibitor of NAMPT is an antibody. As used herein, the term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. The term includes antibody fragments that comprise an antigen binding domain such as Fab′, Fab, F(ab′)2, single domain antibodies (DABs), TandAbs dimer, Fv, scFv (single chain Fv), dsFv, ds-scFv, Fd, linear antibodies, minibodies, diabodies, bispecific antibody fragments, bibody, tribody (scFv-Fab fusions, bispecific or trispecific, respectively); sc-diabody; kappa(lamda) bodies (scFv-CL fusions); BiTE (Bispecific T-cell Engager, scFv-scFv tandems to attract T cells); DVD-Ig (dual variable domain antibody, bispecific format); SIP (small immunoprotein, a kind of minibody); SMIP (“small modular immunopharmaceutical” scFv-Fc dimer; DART (ds-stabilized diabody “Dual Affinity ReTargeting”); small antibody mimetics comprising one or more CDRs and the like. The techniques for preparing and using various antibody-based constructs and fragments are well known in the art (see Kabat et al., 1991, specifically incorporated herein by reference). Diabodies, in particular, are further described in EP 404, 097 and WO 93/11161; whereas linear antibodies are further described in Zapata et al. (1995). Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, Fv, dsFv, Fd, dAbs, TandAbs, ds-scFv, dimers, minibodies, diabodies, bispecific antibody fragments and other fragments can also be synthesized by recombinant techniques or can be chemically synthesized. Techniques for producing antibody fragments are well known and described in the art. For example, each of Beckman et al., 2006; Holliger & Hudson, 2005; Le Gall et al., 2004; Reff & Heard, 2001; Reiter et al., 1996; and Young et al., 1995 further describe and enable the production of effective antibody fragments. In some embodiments, the antibody is a “chimeric” antibody as described in U.S. Pat. No. 4,816,567. In some embodiments, the antibody is a humanized antibody, such as described U.S. Pat. Nos. 6,982,321 and 7,087,409. In some embodiments, the antibody is a human antibody. A “human antibody” such as described in U.S. Pat. Nos. 6,075,181 and 6,150,584. In some embodiments, the antibody is a single domain antibody such as described in EP 0 368 684, WO 06/030220 and WO 06/003388.

In a particular embodiment, the inhibitor is a monoclonal antibody. Monoclonal antibodies can be prepared and isolated using any technique that provides for the production of antibody molecules by continuous cell lines in culture. Techniques for production and isolation include but are not limited to the hybridoma technique, the human B-cell hybridoma technique and the EBV-hybridoma technique.

In a particular, the inhibitor is an intrabody having specificity for NAMPT. As used herein, the term “intrabody” generally refer to an intracellular antibody or antibody fragment. Antibodies, in particular single chain variable antibody fragments (scFv), can be modified for intracellular localization. Such modification may entail for example, the fusion to a stable intracellular protein, such as, e.g., maltose binding protein, or the addition of intracellular trafficking/localization peptide sequences, such as, e.g., the endoplasmic reticulum retention. In some embodiments, the intrabody is a single domain antibody. In some embodiments, the antibody according to the invention is a single domain antibody. The term “single domain antibody” (sdAb) or “VHH” refers to the single heavy chain variable domain of antibodies of the type that can be found in Camelid mammals which are naturally devoid of light chains. Such VHH are also called “Nanobody®”. According to the invention, sdAb can particularly be llama sdAb.

For the first time, inventors have shown that the inhibition of NAMPT expression in A375 melanoma cells was also observed with the MEK inhibitors U0126, PD98059 and GSK1120212 (Trametinib) and the ERK inhibitor, SCH77294, which efficiencies were shown on the inhibition of ERK phosphorylation levels.

Accordingly, the method according to the invention, wherein the inhibitors of MEK and ERK are suitable to inhibit the NAMPT expression and activity. Typically, the inhibitors of MEK are selected from the group consisting of but not limited to: U0126, PD98059, Trametinib (also known as GSK1120212); Cobimetinib (also known Cotellic); Binimetinib (also known as ARRY-162). The inhibitors of ERK are selected from the group consisting of but not limited to: Selumetinib (also known as AZD6244); SCH77294.

Inventors have shown that STAT5 inhibitor decreased both basal and BRAFV600E, stimulated NAMPT promoter activity, thereby demonstrating the involvement of STAT5 in BRAFV600E-induced NAMPT transcription. These data demonstrate that the BRAF/ERK pathway regulates NAMPT expression, and consequently NAD+ levels, at a transcriptional level through STAT5 activation.

Accordingly, the method according to the invention, wherein the inhibitors of STAT5 are suitable to inhibit the NAMPT expression and activity. As used herein, the term “STAT5” refers to signal transducer and activator of transcription 5 and is involved in cytosolic signalling and in mediating the expression of specific genes. Typically, the STAT5 inhibitor reduces STAT5 phosphorylation. The inhibitors of STAT5 are selected from the group consisting of but not limited to: CAS 285986-31-4 (a cell-permeable compound that selectively targets the SH2 domain of STAT5); combination of JAK1/2 and PI3K/mTOR inhibitors; or Pimozid (CAS 2062-78-4).

As used herein the terms “administering” or “administration” refer to the act of injecting or otherwise physically delivering a substance as it exists outside the body (e.g., an inhibitor of NAMPT) into the subject, such as by mucosal, intradermal, intravenous, subcutaneous, intramuscular delivery and/or any other method of physical delivery described herein or known in the art. When a disease, or a symptom thereof, is being treated, administration of the substance typically occurs after the onset of the disease or symptoms thereof. When a disease or symptoms thereof, are being prevented, administration of the substance typically occurs before the onset of the disease or symptoms thereof.

By a “therapeutically effective amount” is meant a sufficient amount of inhibitor of NAMPT for use in a method for the treatment of melanoma at a reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well known within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

The NAMPT inhibitors as described above may be combined with pharmaceutically acceptable excipients, and optionally sustained-release matrices, such as biodegradable polymers, to form pharmaceutical compositions. “Pharmaceutically” or “pharmaceutically acceptable” refer to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The pharmaceutical compositions of the present invention for oral, sublingual, subcutaneous, intramuscular, intravenous, transdermal, local or rectal administration, the active principle, alone or in combination with another active principle, can be administered in a unit administration form, as a mixture with conventional pharmaceutical supports, to animals and human beings. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms. Typically, the pharmaceutical compositions contain vehicles which are pharmaceutically acceptable for a formulation capable of being injected.

These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The polypeptide (or nucleic acid encoding thereof) can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active polypeptides in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion. Some variation in dosage will necessarily occur depending on the condition of the subject being treated. The person responsible for administration will, in any event, determine the appropriate dose for the individual subject.

In another aspect, the invention relates to a method for treating a subject suffering from melanoma resistant comprising following steps: i) determining whether a subject is at risk of relapse to a treatment according to the method, and ii) administering to the subject identified as having a risk of relapse a therapeutically effective amount of nicotinamide phosphoribosyl transferase (NAMPT) inhibitor and a therapeutically effective amount of PLX4032.

In one embodiment, the NAMPT inhibitor administered with a therapeutically effective amount of PLX4032 is the small molecule FK866.

The PLX4032 and the NAMPT inhibitor according to the invention are administered to the subject simultaneously, separately or sequentially.

### Method of Screening

A further object of the present invention relates to a method of screening a drug suitable for the treatment of melanoma comprising i) providing a test compound and ii) determining the ability of said test compound to inhibit the activity of NAMPT.

Any biological assay well known in the art could be suitable for determining the ability of the test compound to inhibit the activity of NAMPT. In some embodiments, the assay first comprises determining the ability of the test compound to bind to NAMPT. In some embodiments, a population of cells is then contacted and activated so as to determine the ability of the test compound to inhibit the activity of NAMPT. In particular, the effect triggered by the test compound is determined relative to that of a population of immune cells incubated in parallel in the absence of the test compound or in the presence of a control agent either of which is analogous to a negative control condition. The term “control substance”, “control agent”, or “control compound” as used herein refers a molecule that is inert or has no activity relating to an ability to modulate a biological activity or expression. It is to be understood that test compounds capable of inhibiting the activity of NAMPT, as determined using in vitro methods described herein, are likely to exhibit similar modulatory capacity in applications in vivo. Typically, the test compound is selected from the group consisting of peptides, peptidomimetics, small organic molecules, aptamers or nucleic acids. For example the test compound according to the invention may be selected from a library of compounds previously synthesised, or a library of compounds for which the structure is determined in a database, or from a library of compounds that have been synthesised de novo. In some embodiments, the test compound may be selected form small organic molecules.

The invention will be further illustrated by the following figures and examples. However, these examples and figures should not be interpreted in any way as limiting the scope of the present invention.

### FIGURES

FIG. 1: PLX4032 decreases the levels of NAD+ in BRAFV600E melanoma cells.

(A) Scatter plot showing the means+/−SD of the normalized NAD+ values in control and PLX4032-treated WM9 and UACC62 melanoma cells. (B) Intracellular NAD+ levels in a panel of BRAFV600E and WT BRAF human melanoma cells exposed or not to PLX4032 (5 μM, 24 h). The values represent the means+/−SD of five independent experiments.

FIG. 2: BRAF/MEK/ERK signaling pathway regulates nicotinamide phosphoribosyltransferase (NAMPT) expression. (A) NAMPT mRNA levels in BRAFV600E melanoma cells (A375, WM9, and SKme128) were measured using QPCR, under the same conditions. (B-C) Analysis of publicly available data sets GSE42872 and GSE20051 of melanoma cells exposed to PLX4032. Scatter plots showing the means+/−SD of the NAMPT mRNA expression are shown.

FIG. 3: BRAF regulates NAMPT transcription through STAT5.

(A-B) Activity of NAMPT promoter reporter in A375 and WM9 cells respectively exposed to increasing doses of PLX4032 (5 μM, 48 h). Data are shown as the means+/−SD of 4 experiments. For A375, all the PLX4032 doses lead to significant decrease in NAMPT promoter activity (p<0.0034). For WM9, significant decrease in NAMPT promoter activity is observed with PLX4032>0.005, (p<0.014). (C) Activity of NAMPT promoter reporter vector in A375 cells exposed to MEK (GSK1120212 and U0126) or ERK (SCH772984) inhibitors. (D) Activity of NAMPT promoter segments with different lengths in A375 cells exposed to PLX4032. (E) Activity of NAMPT promoter reporter in 501MEL cells transfected with a control vector or a vector encoding BRAFV600E and left untreated or exposed to STAT5 inhibitor (STAT5i, 100 μM, 48 h). For A to E, histograms (means+/−SD, n=2, excepted for E, n=4) of relative luciferase activity, normalized to β-galactosidase are shown. (F) Histograms (means+/−SD, n=6) showing the NAD+ level in A375 and WM9 cells exposed to STAT5 inhibitor.

FIG. 4: NAMPT is required for melanoma proliferation and tumor development.

(A) Intracellular NAD+ levels in A375 and WM9 melanoma cells are treated with control siRNA or 2 different NAMPT siRNA or exposed to NAMPT inhibitor (FK866). HSP90 was used as loading control. Values represent the means+/−SD of three independent experiments. (B) Proliferation of WM9 and A375 melanoma cells treated as in (A). Cells were trypsinized and counted each day. Values represent the means+/−SD of three independent experiments. (C) Growth curve of tumor xenografts after subcutaneous injection of WM9 cells. Mice (6 per group) were treated or not with PXL4032 or FK866. Data are shown as the means+/−SD of tumor volume. Black arrow indicates beginning of the treatment.

FIG. 5: NAMPT affects the sensitivity of melanoma cells to BRAF inhibitor.

(A) Intracellular NAD+ level in A375 and WM9 melanoma cells transduced with a control or NAMPT adenovirus. Values represent the means+/−SD of three independent experiments. (B) A375 and WM9 melanoma cells transduced with a control or NAMPT adenovirus were exposed to PLX4032 (5 μM). After 72 h, the cells were counted. The histogram represents the means+/−SD of 3 independent experiments. (C) A375 melanoma cells resistant to PLX4032 (A375R) were transfected with control or NAMPT siRNA (si #1 and si #2) and subsequently exposed to PLX4032 (5 μM). After 48 h, the cells were counted. The histogram represents the means+/−SD of 3 independent experiments. (D) WM9 melanoma cells resistant to PLX4032 (WM9R) were transfected with control or NAMPT siRNA (si #1 and si #2) and subsequently exposed to PLX4032 (5 μM). After 72 h, the cells were counted. The histogram represents the means+SD of three independent experiments. (E) Growth curve of tumor xenografts of PLX4032-resistant A375 melanoma cells (A375R) after subcutaneous injection). Mice (6 per group) were treated with vehicle, PLX4032 (25 mg/kg) or FK866 (1.5 mg/kg and 15 mg/kg) alone or with the low FK866 dose in combination with PLX4032. Data are presented as the means+/−SD. Black arrow indicates beginning of the treatment. (F) Growth curve of tumor xenografts after subcutaneous injection of WM9 cells resistant to PLX4032 (WM9R). Mice (6 per group) were treated with vehicle, PLX4032 or FK866. Data are shown as the means+/−SD of tumor volume. Black arrow indicates beginning of the treatment.

### EXAMPLE

Material & Methods

Cell Cultures and Reagents

Human melanoma cell lines and short-term cultures derived from different patients with metastatic malignant melanoma cells were grown in DMEM supplemented with 7% FBS at 37° C. in a humidified atmosphere containing 5% CO2. PLX4032-sensitive and PLX4032-resistant melanoma cells were previously described (Bonet et al. 2012; Ohanna et al. 2014). Lipofectamine™ RNAiMAX and opti-MEM medium were purchased from Invitrogen (San Diego, Calif., USA). FK866 was obtained from Sigma, U0126 and GSK1120212 were purchased from Euromedex, and PD98059 and SCH77294 were obtained from Selleck Chemicals. The STAT5 inhibitor (CAS 285986-31-4) was purchased from Sigma. Intracellular NAD+ was measured using the NAD/NADH Quantitation Kit from Sigma according to the manufacturer's instructions.

Metabolomic Profiling

Briefly, samples were prepared using the automated MicroLab STAR® system (Hamilton Company). Recovery standards were added prior to the first step in the extraction process for QC purposes. Cell lysates were precipitated using methanol under vigorous shaking for 2 min, followed by centrifugation. The resulting extract was divided into five fractions: two samples for analysis using two separate reversephase (RP)/UPLC-MS/MS methods with positive ion mode electrospray ionization (ESI), one sample for analysis through RP/UPLC-MS/MS with negative ion mode ESI, one sample for analysis through HILIC/UPLC-MS/MS with negative ion mode ESI, and one sample was reserved for backup. The samples were briefly placed on a TurboVap® (Zymark) to remove the organic solvent. The sample extracts were stored overnight under nitrogen prior to preparation for analysis. Chromatography analyses were performed using Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried and subsequently reconstituted in solvents compatible to each method (Miller et al. 2015). The informatics system comprised four major components, the Laboratory Information Management System (LIMS), the data extraction and peak identification software, data processing tools for QC and compound identification, and a collection of information interpretation and visualization tools for subsequent data analysis. The hardware and software foundations for these informatics components were the LAN backbone and a database server running Oracle 10.2.0.1 Enterprise Edition (Evans et al. 2009).

Western Blot Assays

Western blotting was performed as previously described (Hilmi et al. 2008; Bertolotto et al. 2011). Briefly, cell lysates (30 μg) were separated using SDS-PAGE, transferred onto a PVDF membrane and subsequently exposed to the appropriate antibodies, anti-ERK2 (Santa Cruz Biotechnology, sc-1647 clone D-2), anti-phospho-ERK1/2 (Thr202/Tyr204) (Cell Signaling Technology Inc., #2370), anti-BRAF (Santa Cruz Biotechnology, sc-5284 clone F-7), anti-NAMPT (Sigma, #B5812), antiphospho-STAT5 (Tyr694) (Ozyme #9351), anti-STAT5 (Ozyme #9363), and anti-HSP90 (Santa Cruz biotechnology, #sc-13119). The proteins were visualized using the ECL system (Amersham). The western blots shown are representative of at least 3 independent experiments.

Transient Transfection of siRNA

Briefly, a single pulse of 50 nM of siRNA was administered to the cells at 50% confluency through transfection with 5 μl of Lipofectamine™ RNAiMAX in Opti-MEM medium (Invitrogen, San Diego, Calif., USA). NAMPT siRNAs (ON TARGET plus, Dharmacon) were obtained from Thermo Fisher Scientific.

Cell Proliferation.

The cells were seeded onto 12-well dishes (10×103 cells), and at 48 h post transfection or treatment, the cells were trypsinized from days 1 to 4, counted in triplicate using a hemocytometer. The experiments were performed at least three times.

Luciferase Reporter Assays

NAMPT promoter luciferase reporters were provided by Dr. J.G.N. Garcia (University of Arizona). We used 3 constructs containing the following regions of human NAMPT: −2682/+346; −1182/+346; and −582/+346 base pairs (Sun et al. 2014). A375 melanoma cells were transiently transfected as previously described using Lipofectamine reagent (Invitrogen) (Bertolotto et al. 1998). Briefly, the cells were transiently transfected with 0.3 μg of NAMPT reporter constructs and 0.05 μg of pCMVBGal to control the variability in transfection efficiency. The transfection medium was changed after 6 h, and where indicated, the cells were transfected with an empty vector or a vector encoding BRAFV600E or treated with PLX4032 or STAT5 inhibitor. The cells were assayed for luciferase and β-galactosidase activities after 48 h. The experiments were repeated at least three times.

Colony Formation Assay

Human melanoma cells were seeded onto 6-well plates. The cells were subsequently placed in a 37° C., 5% CO2 incubator. Colonies of cells were grown before being stained with 0.04% crystal violet/2% ethanol in PBS for 30 min. Photographs of the stained colonies were captured. The colony formation assay was performed in duplicate.

Cell Death Analysis by Flow Cytometry

Cells were seeded at a density of 50 000 cells/well, in 24-well plate and treated with FK866 for indicated time. Cells were harvested using Accutase enzyme, washed twice with ice-cold phosphate-buffered saline, resuspended in medium with DAPI (1 μg/ml) and incubated for 15 minutes at room temperature (25° C.) in the dark. Samples were immediately analyzed by a flow cytometer (MACS QUANT) using a laser at 405 nm excitation with a bandpass filter at 425 nm and 475 nm for DAPI detection.

mRNA Preparation and Real-Time/Quantitative PCR

The mRNA was isolated using TRIzol (Invitrogen) according to a standard procedure. QRT-PCR was performed using SYBR® Green I (Eurogentec, Seraing, Belgium) and Multiscribe Reverse Transcriptase (Applied Biosystems) and subsequently monitored using the ABI Prism 7900 Sequence Detection System (Applied Biosystems, Foster City, Calif.). The detection of the SB34 gene was used to normalize the results. Primer sequences for each cDNA were designed using either Primer Express Software (Applied Biosystems) or qPrimer depot (http://primerdepot.nci.nih.gov), and these sequences are available upon request.

Animal Experimentation

Animal experiments were performed in accordance with French law and approved by a local institutional ethical committee. The animals were maintained in a temperature controlled facility (22° C.) on a 12-h light/dark cycle and provided free access to food (standard laboratory chow diet from UAR, Epinay-S/Orge, France). Human WM9 melanoma cells, responsive or resistant to PLX4032 (2×106 cells), were subcutaneously inoculated into 8-week-old female, immune-deficient, athymic, nude FOXN1nu mice (Harlan Laboratory). When the tumors became palpable (0.1-0.2 cm3), the mice received an intraperitoneal injection of PLX4032 (25 mg/kg), FK866 (15 mg/kg) or both drugs dissolved in a mixture of Labrafil M1944 Cs, dimethylacetamide, and Tween 80 (90:9:1, v/v/v) three times per week. Control mice were injected with Labrafil alone. The growth tumor curves were determined after measuring the tumor volume using the equation V=(L×W2)/2. At the end of the experiment, the mice were euthanized by cervical dislocation, and the tumors were harvested for immunofluorescence.

Immunofluorescence Studies

Frozen sections of melanoma xenografts were fixed with 4% paraformaldehyde (PFA, Sigma-Aldrich) for 15 min and subsequently blocked with 10% normal goat serum (Vector) with or without 0.1% Triton X-100 (Bio-Rad) in PBS for 30 min at room temperature. The samples were incubated with primary antibodies overnight at +4° C. followed by the appropriate secondary fluorescent-labeled antibodies (Invitrogen Molecular Probes) for 1 h at room temperature and mounted using Gel/Mount (Biomeda Corp., Foster City, Calif.). The nuclei were counterstained with DAPI. Apoptosis in melanoma xenografts was detected through a TUNEL assay using an in-situ cell apoptosis kit (R&D Systems). Immunofluorescence was examined and photographed using a Zeiss Axiophot microscope equipped with epifluorescence illumination.

Statistical Analysis

The data are presented as the means±SD and analyzed using two-sided Student's test with Prism or Microsoft Excel software. The difference between both conditions was statistically significant at p<0.05. For the metabolomics analysis, the p values were adjusted using the Benjamini-Hochberg procedure (Anastats). Supplementary information is available at Cell research's website.

Results

Alteration of Metabolism by PLX4032 in BRAFV600E Melanoma Cells.

To fully elucidate the effect of PLX4032 on the metabolism of melanoma cells, we performed global metabolic profiling using Ultrahigh Performance Liquid Chromatography-Tandem Mass Spectroscopy of two distinct human melanoma cell lines (UACC62 and WM9) that harbor the BRAFV600E mutation. Analysis of more than 500 biochemicals identified 93 metabolites altered by treatment with PLX4032. A total of 45 metabolites were downregulated, whereas 48 metabolites were upregulated. The heat map shows the 40 most significantly regulated metabolites. Further analysis identified (data not shown) the downregulation of 4 biochemicals in glycolysis and 3 compounds in the TCA cycle indicating the inhibition of the carbohydrate metabolism and energy production. Twelve dipeptides, 6 monoacylglycerols, 6 long chain fatty acids (PUFA) and 4 sphingolipids were upregulated upon PLX4032 treatment, suggesting a global increase in protein catabolism and lipid synthesis. In addition, 6 metabolites in the methionine, cysteine and taurine pathways and 7 in the purine metabolic pathway were either up or down regulated by PLX4032. A schematic view of the metabolic consequences of BRAF inhibition is provided (data not shown).

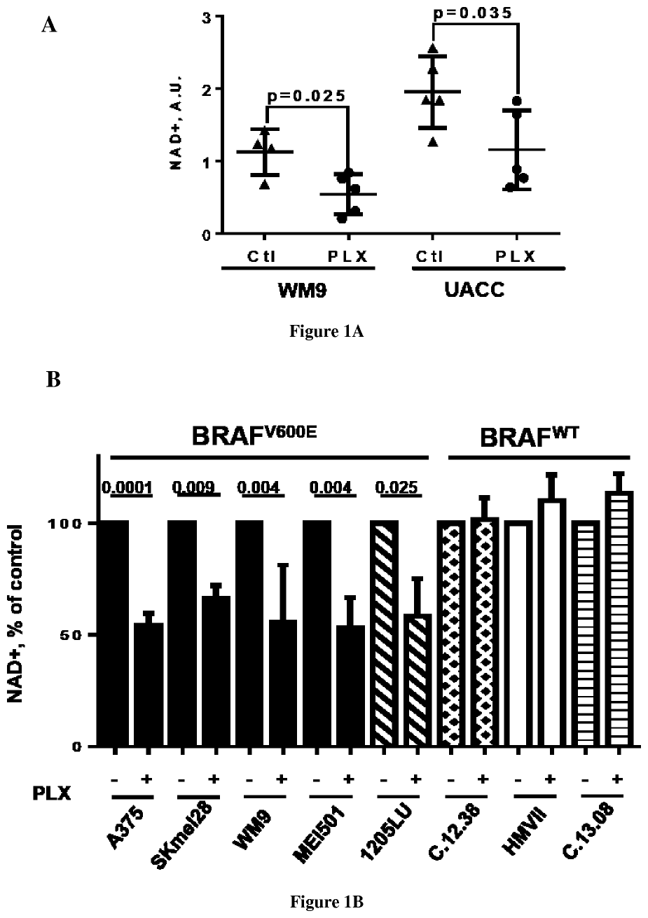

Among the other metabolites, we observed that nicotinamide adenine dinucleotide (NAD+) levels were reduced in both melanoma cell lines exposed to PLX4032 (FIG. 1A). We extended these analyses to a larger panel of melanoma cell lines and short-term melanoma cell cultures. As expected, PLX4032 inhibited cell proliferation in BRAFV600E but not in BRAFWT human melanoma cells (data not shown). Importantly, PLX4032 reduced intracellular NAD+ levels in all BRAFV600E-mutated melanoma cell lines and short-term melanoma cell cultures but not in BRAFWT human melanoma cells (FIG. 1B). Further, additional BRAF inhibitors as well as MEK and ERK inhibitors also decreased NAD+ levels, indicating that ERK pathway inhibition impacted NAD+ metabolism in A375 melanoma cells (data not shown).

BRAFV600E Regulates NAD+ Levels and Controls NAMPT Expression.

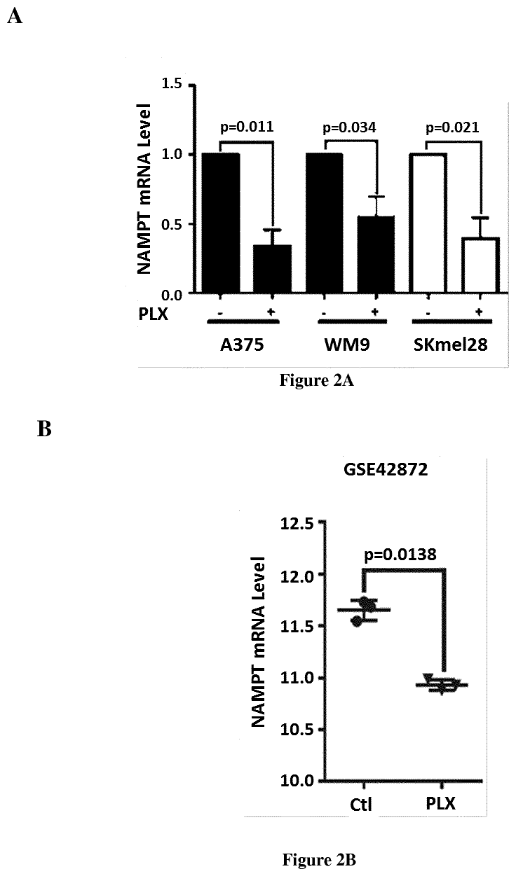

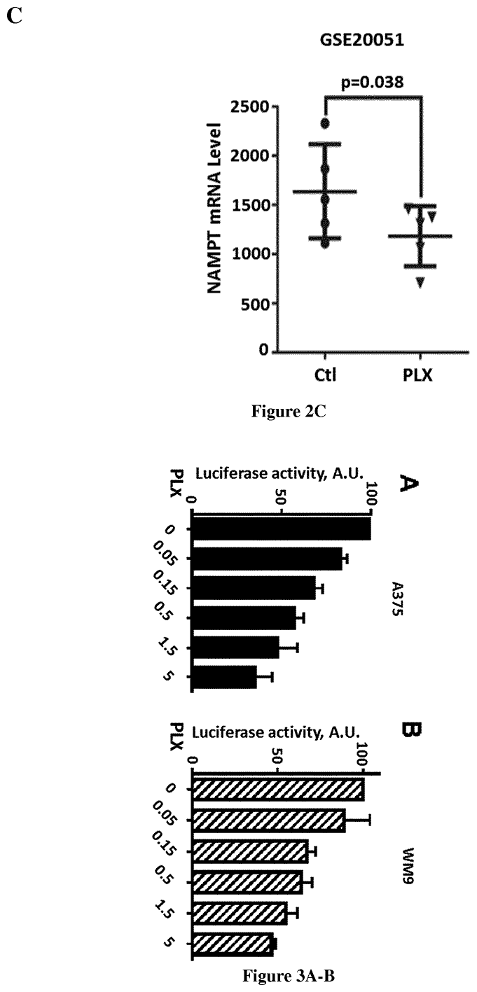

The essential of NAD+ as co-factor for multiple metabolic pathways, prompted us to investigate the mechanism by which PLX4032 regulate the level of NAD+ in melanoma cells. The NAD+ level is primarily maintained in human cells via the “salvage” pathway in which NAMPT is the rate-limiting enzyme (Canto et al. 2015). Our results indicated that PLX4032 reduced NAMPT expression at both the protein (data not shown) and mRNA levels (FIG. 2A) in 4 different BRAFV600E melanoma cells. No inhibition of NAMPT expression by PLX4032 was observed in BRAFWT human melanoma cells (data not shown). Analysis of publicly available microarray data sets (GSE20051 and GSE42872) from other sources confirmed the inhibition of NAMPT mRNA expression in BRAFV600E melanoma cell lines exposed to BRAF inhibitor (FIGS. 2B-2C). The inhibition of NAMPT expression in A375 melanoma cells was also observed with the MEK inhibitors U0126, PD98059 and GSK1120212 (Trametinib) and the ERK inhibitor, SCH77294, which efficiencies were shown on the inhibition of ERK phosphorylation levels (data not shown). We also observed the inhibition of NAMPT expression in publicly available microarray data (GSE51115) of melanoma cell lines exposed to the MEK inhibitor, PD0325901 (data not shown). Additionally, forced expression of BRAFV600E in normal human melanocytes stimulated the ERK signaling pathway and increased levels of NAMPT (data not shown), which were prevented using MEK and ERK inhibitors. Altogether, our data demonstrate that the BRAF/MEK/ERK signaling cascade plays a key role in the control of NAMPT expression and the regulation of NAD+ metabolism in melanoma cells.

Regulation of NAMPT Transcription Through the BRAF-STAT5 Signaling Cascade.

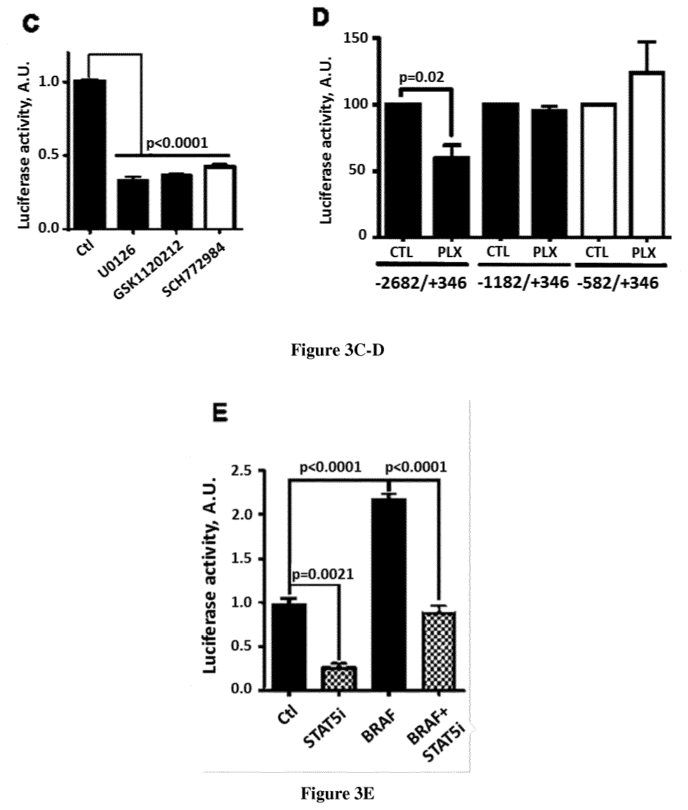

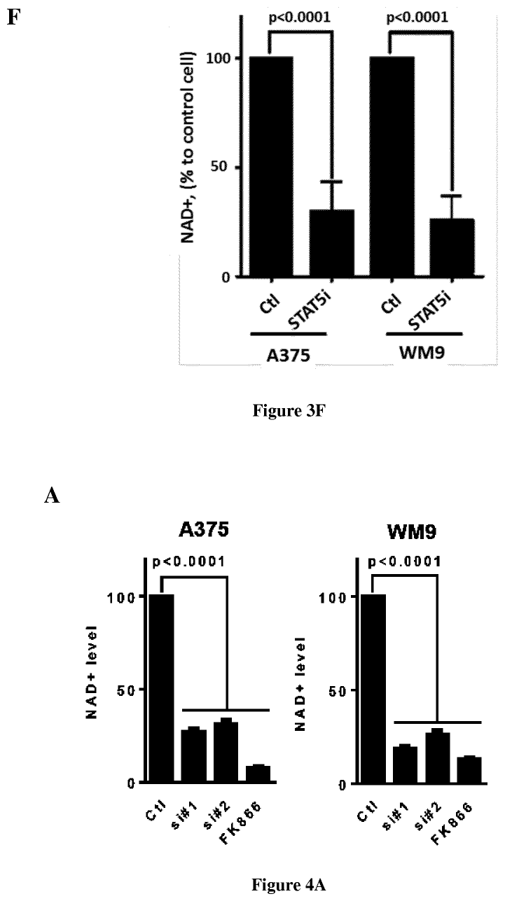

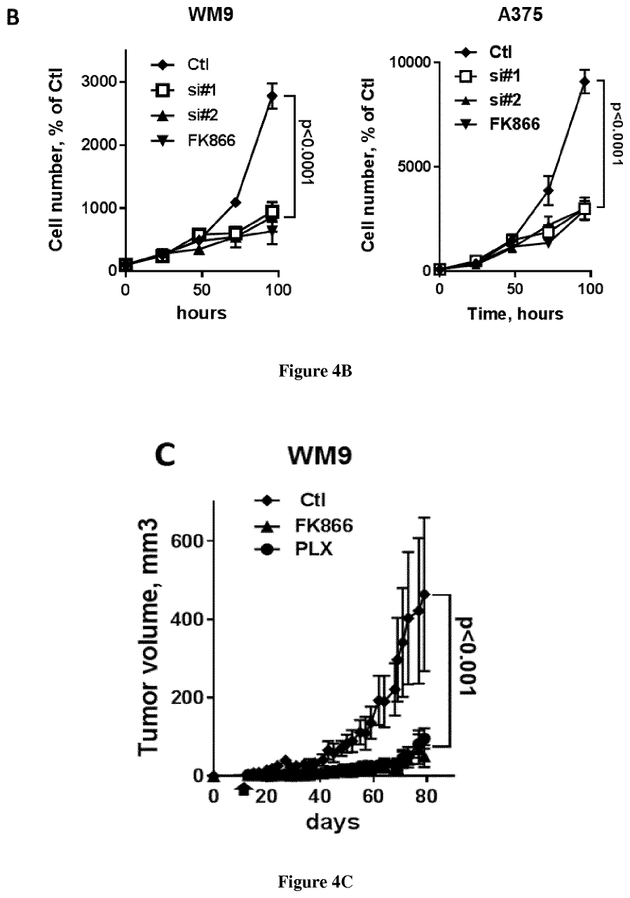

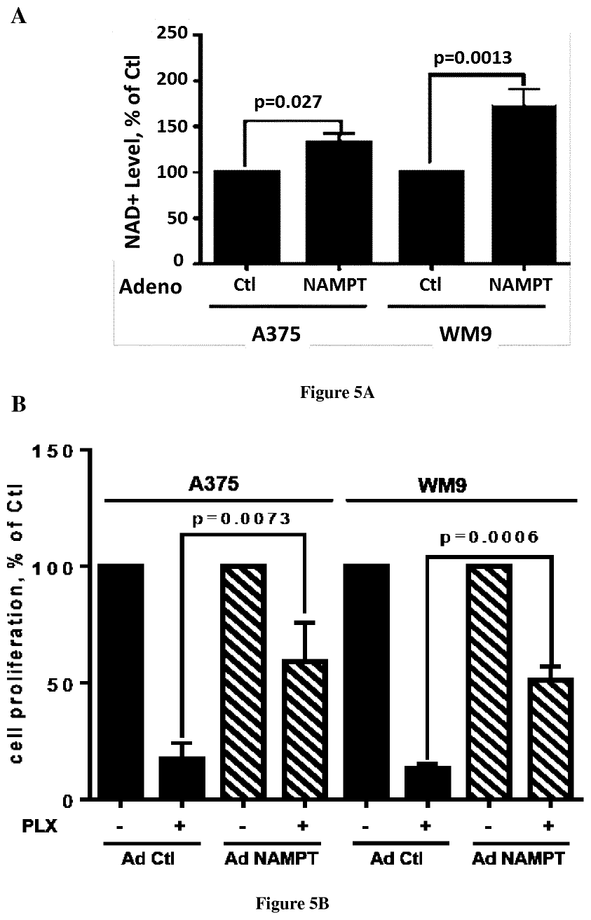

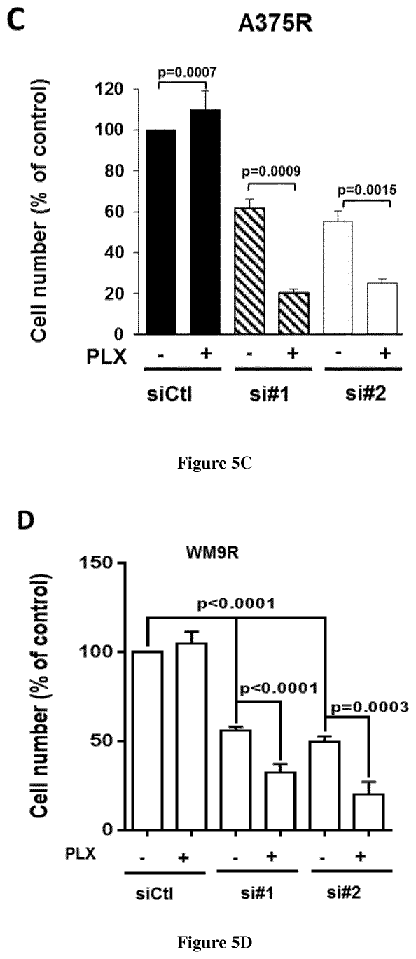

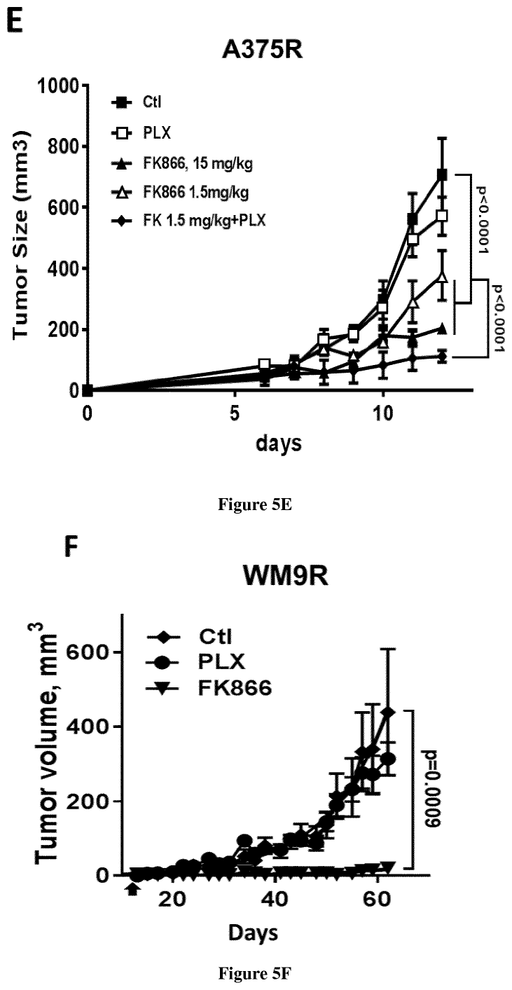

Changes in NAMPT mRNA levels suggested that the BRAF/ERK pathway controlled NAMPT at the transcriptional level. Using a human NAMPT promoter luciferase reporter construct, we showed that PLX4032 induced a dose-dependent decrease in NAMPT promoter activity in both A375 and WM9 cells (FIGS. 3A-B). MEK and ERK inhibitors also strongly reduced NAMPT promoter activity (FIG. 3C). To identify the regulatory elements, we assessed the effect of PLX4032 on human NAMPT promoter constructs of different lengths. The results revealed that the BRAF/ERK responsive element was localized between 1182 and 2682 base pairs upstream of the transcriptional start site (FIG. 3D). Within this region, Sun et al. identified a STAT5 site that mediated the mechanical stress signal to upregulate NAMPT gene transcription (Sun et al. 2014). Therefore, we hypothesized that in melanoma cells, the ERK pathway might control NAMPT expression through the activation of STAT5. We showed that STAT5 inhibitor decreased both basal and BRAFV600E-stimulated NAMPT promoter activity, thereby demonstrating the involvement of STAT5 in the BRAFV600E-induced stimulation of NAMPT transcription (FIG. 3E). In both A375 and WM9 cells, we observed that PLX4032 inhibited both ERK and STAT5 phosphorylation, while STAT5 inhibitor efficiently reduced STAT5 phosphorylation but did not affect ERK phosphorylation (FIG. 3F). Both BRAF and STAT5 inhibitors decreased NAMPT expression (data not shown) and NAD+ levels (FIG. 1D and FIG. 3F). Taken together, these data demonstrate that the BRAF/ERK pathway regulates NAMPT expression, and consequently NAD+ levels, at the transcriptional level through STAT5 activation.

NAMPT Controls Melanoma Cell Proliferation.

To determine the impact of NAD+ metabolism on melanoma cell proliferation, we used FK866, a highly specific non-competitive inhibitor of NAMPT, and NAMPT siRNA. As expected, 2 different NAMPT siRNAs efficiently inhibited NAMPT expression in both A375 and WM9 cells (data not shown). FK866 did not affect NAMPT expression. Both FK866 and NAMPT siRNAs dramatically decreased the intracellular levels of NAD+, in both A375 and WM9 melanoma cell lines (FIG. 4A) and impaired cell proliferation (FIG. 4B). Interestingly, the addition of exogenous NAD+ to the culture medium reversed the effects of FK866, thereby demonstrating that the effect of FK866 was causally associated with NAD+ depletion and not to non-specific effects (data not shown). Further, flow cytometry analysis of DAPI permeable cells showed that NAMPT inhibition by FK866 or siRNA increased cell death (data not shown). We next monitored the effect of FK866 in vivo. WM9 cells were subcutaneously engrafted into 6-week-old female athymic nude mice. When the tumors became palpable (0.1-0.2 cm3), the mice were treated with FK866 at a dose of 15 mg/kg or with its vehicle (labrafil) every two days. Compared with vehicle treatment, FK866 treatment impaired WM9 melanoma cell xenograft growth (FIG. 4C). FK866 caused no weight loss in the treated mice (not shown). Thus, the inhibition of NAMPT has clear anti-melanoma effects in vitro and in vivo.

NAMPT Affects the Sensitivity of Melanoma Cells to BRAF Inhibitor.

We next examined whether NAMPT might affect the response to PLX4032. Forced expression of NAMPT (data not shown) enhanced the intracellular NAD+ level (FIG. 5A) but did not significantly affect proliferation (data not shown). As expected, PLX4032 inhibited the viability of A375 and WM9 melanoma cells. Forced expression of NAMPT markedly decreased the efficiency of PLX4032 to inhibit the proliferation of melanoma cells (FIG. 5B). These observations prompted us to hypothesize that NAMPT inhibition might impact the sensitivity of BRAFi-resistant melanoma cells to PLX4032. In agreement with this hypothesis, we observed that NAMPT silencing strongly inhibited the proliferation of BRAFi-resistant A375 melanoma cells (A375R) and BRAFi-resistant WM9 melanoma cells (WM9R) (FIG. 5C and FIG. 5D). Interestingly, although NAMPT inhibition had a marked effect on BRAFi-resistant cells, we observed the statistically significant restoration of PLX4032 sensitivity in BRAFi-resistant cells, upon depletion of NAMPT (FIG. 5C and FIG. 5D). In vivo, xenografts derived from BRAFi-resistant WM9 melanoma cells were not affected by PLX4032 but were highly sensitive to NAMPT inhibition by FK866: FK866 inhibited tumor growth (FIG. 5F). We also performed in vivo experiments on A375R cells. In contrast to PLX4032, increasing doses of FK866 dose-dependently reduced melanoma progression (FIG. 5E). As expected, a reduction in NAD⁺ was observed in the FK866-treated tumors (data not shown). The combination of both PLX4032 and FK866 resulted in a synergistic inhibition of melanoma growth. Thus, both in vitro and in vivo, FK866 treatment caused melanoma cell death and restored PLX4032 sensitivity in BRAFi-resistant melanoma cells.

Nevertheless, the striking effect of NAMPT inhibition on BRAFi-resistant melanoma xenografts suggests that targeting NAMPT might be a valid therapeutic option to overcome BRAFi resistance.

### REFERENCES

Throughout this application, various references describe the state of the art to which this invention pertains. The disclosures of these references are hereby incorporated by reference into the present disclosure.
Baenke F, Chaneton B, Smith M, Van Den Broek N, Hogan K, Tang H, Viros A, Martin M, Galbraith L, Girotti M R et al. 2016. Resistance to BRAF inhibitors induces glutamine dependency in melanoma cells. Molecular oncology 10: 73-84.
Bai P, Canto C. 2012. The role of PARP-1 and PARP-2 enzymes in metabolic regulation and disease. Cell metabolism 16: 290-295.
Balasubramanian P, Zhao L J, Chinnadurai G. 2003. Nicotinamide adenine dinucleotide stimulates oligomerization, interaction with adenovirus E1A and an intrinsic dehydrogenase activity of CtBP. FEBS Lett 537: 157-160.
Baudy A R, Dogan T, Flores-Mercado J E, Hoeflich K P, Su F, van Bruggen N, Williams S P. 2012. FDG-PET is a good biomarker of both early response and acquired resistance in BRAFV600 mutant melanomas treated with vemurafenib and the MEK inhibitor GDC-0973. EJNMMI research 2: 22.
Bertolotto C, Abbe P, Hemesath T J, Bille K, Fisher D E, Ortonne J P, Ballotti R. 1998.
Microphthalmia gene product as a signal transducer in cAMP-induced differentiation of melanocytes. J Cell Biol 142: 827-835.
Bertolotto C, Lesueur F, Giuliano S, Strub T, de Lichy M, Bille K, Dessen P, d'Hayer
B, Mohamdi H, Remenieras A et al. 2011. A SUMOylation-defective MITF germline mutation predisposes to melanoma and renal carcinoma. Nature 480: 94-98.
Bonet C, Giuliano S, Ohanna M, Bille K, Allegra M, Lacour J P, Bahadoran P, Rocchi S, Ballotti R, Bertolotto C. 2012. Aurora B is regulated by the mitogenactivated protein kinase/extracellular signal-regulated kinase (MAPK/ERK) signaling pathway and is a valuable potential target in melanoma cells. J Biol Chem 287: 29887-29898.
Canto C, Menzies K J, Auwerx J. 2015. NAD(+) Metabolism and the Control of Energy Homeostasis: A Balancing Act between Mitochondria and the Nucleus. Cell metabolism 22: 31-53.
Delgado-Goni T, Miniotis M F, Wantuch S, Parkes H G, Marais R, Workman P, Leach M O, Beloueche-Babari M. 2016. The BRAF Inhibitor Vemurafenib Activates Mitochondrial Metabolism and Inhibits Hyperpolarized Pyruvate-Lactate Exchange in BRAF-Mutant Human Melanoma Cells. Mol Cancer Ther 15: 2987-2999.
Evans A M, DeHaven C D, Barrett T, Mitchell M, Milgram E. 2009. Integrated, nontargeted ultrahigh performance liquid chromatography/electrospray ionization tandem mass spectrometry platform for the identification and relative quantification of the small-molecule complement of biological systems. Analytical chemistry 81: 6656-6667.
Grolla A A, Torretta S, Gnemmi I, Amoruso A, Orsomando G, Gatti M, Caldarelli A, Lim D, Penengo L, Brunelleschi S et al. 2015. Nicotinamide phosphoribosyltransferase (NAMPT/PBEF/visfatin) is a tumoural cytokine released from melanoma. Pigment Cell Melanoma Res 28: 718-729.
Haq R, Shoag J, Andreu-Perez P, Yokoyama S, Edelman H, Rowe G C, Frederick D T, Hurley A D, Nellore A, Kung A L et al. 2013. Oncogenic BRAF regulates oxidative metabolism via PGClalpha and MITF. Cancer Cell 23: 302-315.
Hassel J C, Winnemoller D, Schartl M, Wellbrock C. 2008. STAT5 contributes to antiapoptosis in melanoma. Melanoma Res 18: 378-385.
Hilmi C, Larribere L, Giuliano S, Bille K, Ortonne J P, Ballotti R, Bertolotto C. 2008.
IGF1 promotes resistance to apoptosis in melanoma cells through an increased expression of BCL2, BCL-X(L), and survivin. J Invest Dermatol 128: 1499-1505.
Holen K, Saltz L B, Hollywood E, Burk K, Hanauske A R. 2008. The pharmacokinetics, toxicities, and biologic effects of FK866, a nicotinamide adenine dinucleotide biosynthesis inhibitor. Investigational new drugs 26: 45-51.
Houtkooper R H, Pirinen E, Auwerx J. 2012. Sirtuins as regulators of metabolism and healthspan. Nat Rev Mol Cell Biol 13: 225-238.
Kennedy B E, Sharif T, Martell E, Dai C, Kim Y, Lee P W, Gujar S A. 2016. NAD+ salvage pathway in cancer metabolism and therapy. Pharmacological research: the official journal of the Italian Pharmacological Society 114: 274-283.
Liang H, Fekete D M, Andrisani O M. 2011. CtBP2 downregulation during neural crest specification induces expression of Mitf and REST, resulting in melanocyte differentiation and sympathoadrenal lineage suppression. Mol Cell Biol 31: 955-970.
Luke J J, Flaherty K T, Ribas A, Long G V. 2017. Targeted agents and immunotherapies: optimizing outcomes in melanoma. Nature reviews Clinical oncology 14: 463-482.
Maki K, Ikuta K. 2008. MEK1/2 induces STAT5-mediated germline transcription of the TCRgamma locus in response to IL-7R signaling. J Immunol 181: 494-502.
Maldi E, Travelli C, Caldarelli A, Agazzone N, Cintura S, Galli U, Scatolini M, Ostano P, Miglino B, Chiorino G et al. 2013. Nicotinamide phosphoribosyltransferase (NAMPT) is over-expressed in melanoma lesions. Pigment Cell Melanoma Res 26: 144-146.
Miller M J, Kennedy A D, Eckhart A D, Burrage L C, Wulff J E, Miller L A, Milburn M V,
Ryals J A, Beaudet A L, Sun Q et al. 2015. Untargeted metabolomic analysis for the clinical screening of inborn errors of metabolism. Journal of inherited metabolic disease 38: 1029-1039.
Mirmohammadsadegh A, Hassan M, Bardenheuer W, Marini A, Gustrau A, Nambiar S, Tannapfel A, Bojar H, Ruzicka T, Hengge U R. 2006. STAT5 phosphorylation in malignant melanoma is important for survival and is mediated through SRC and JAK1 kinases. J Invest Dermatol 126: 2272-2280.
Ohanna M, Bonet C, Bille K, Allegra M, Davidson I, Bahadoran P, Lacour J P, Ballotti R, Bertolotto C. 2014. SIRT1 promotes proliferation and inhibits the senescence-like phenotype in human melanoma cells. Oncotarget 5: 2085-2095.
Parmenter T J, Kleinschmidt M, Kinross K M, Bond S T, Li J, Kaadige M R, Rao A, Sheppard K E, Hugo W, Pupo G M et al. 2014. Response of BRAF-mutant melanoma to BRAF inhibition is mediated by a network of transcriptional regulators of glycolysis. Cancer discovery 4: 423-433.
Ravaud A, Cerny T, Terret C, Wanders J, Bui B N, Hess D, Droz J P, Fumoleau P, Twelves C. 2005. Phase I study and pharmacokinetic of CHS-828, a guanidino-containing compound, administered orally as a single dose every 3 weeks in solid tumours: an ECSG/EORTC study. Eur J Cancer 41: 702-707.
Rodriguez M I, Peralta-Leal A, O'Valle F, Rodriguez-Vargas J M, Gonzalez-Flores A, Majuelos-Melguizo J, Lopez L, Serrano S, de Herreros A G, Rodriguez-Manzaneque J C et al. 2013. PARP-1 regulates metastatic melanoma through modulation of vimentin-induced malignant transformation. PLoS genetics 9: e1003531.
Sun X, Elangovan V R, Mapes B, Camp S M, Sammani S, Saadat L, Ceco E, Ma S F, Flores C, MacDougall M S et al. 2014. The NAMPT promoter is regulated by mechanical stress, signal transducer and activator of transcription 5, and acute respiratory distress syndrome-associated genetic variants. American journal ofrespiratory cell and molecular biology 51: 660-667.
Tateishi K, Wakimoto H, Iafrate A J, Tanaka S, Loebel F, Lelic N, Wiederschain D, Bedel O, Deng G, Zhang B et al. 2015. Extreme Vulnerability of IDH1 Mutant Cancers to NAD+ Depletion. Cancer Cell 28: 773-784.
Tripathi P, Kurtulus S, Wojciechowski S, Sholl A, Hoebe K, Morris S C, Finkelman F D, Grimes H L, Hildeman D A. 2010. STAT5 is critical to maintain effector CD8+ T cell responses. J Immunol 185: 2116-2124.
von Heideman A, Berglund A, Larsson R, Nygren P. 2010. Safety and efficacy of NAD depleting cancer drugs: results of a phase I clinical trial of CHS 828 and overview of published data. Cancer chemotherapy and pharmacology 65: 1165-1172.
Wachsman W, Morhenn V, Palmer T, Walls L, Hata T, Zalla J, Scheinberg R, Sofen H, Mraz S, Gross K et al. 2011. Noninvasive genomic detection of melanoma. Br J Dermatol 164: 797-806.
Warburg O. 1956. On the origin of cancer cells. Science 123: 309-314.
Ward P S, Thompson C B. 2012. Metabolic reprogramming: a cancer hallmark even warburg did not anticipate. Cancer Cell 21: 297-308.

1. A method for predicting the risk of relapse to a treatment in a subject suffering from melanoma comprising the steps of: i) measuring the activity and/or expression level of NAMPT in a biological sample obtained from said subject; ii) comparing the expression level measured at step i) with its predetermined reference value, and iii) concluding that the subject is at risk of relapse to the treatment when the expression level of NAMPT is higher than its predetermined reference value or concluding that the subject is not at risk of relapse when the expression level of NAMPT is lower than its predetermined reference value.

2. The method according to claim 1, wherein, the activity of NAMPT is determined by measuring the production level of NAD.

3. The method according to claim 1 comprising the steps of: i) measuring the activity of NAMPT by determining the production level of NAD+ in a biological sample obtained from said subject; ii) comparing the production level of NAD+ measured at step i) with its predetermined reference value, and iii) concluding that the subject is at risk of relapse to the treatment when the production level of NAD+ is higher than its predetermined reference value or concluding that the subject is not at risk of relapse when the production level of NAD+ is lower than its predetermined reference value.

4. The method according to claim 1, wherein the subject is or is susceptible to becoming resistant to the treatment.

5. The method according to claim 1, wherein the treatment is selected from the group consisting of: PLX4032, immunotherapy and combined treatment.

6. A method for treating a subject who has melanoma and is resistant to one or more melanoma treatments or is at risk of developing resistance to one or more melanoma treatments, comprising administering to said subject a therapeutically effective amount of an inhibitor of nicotinamide phosphoribosyl transferase (NAMPT).

7. The method according to claim 6, comprising, prior to the step of administering, determining whether the subject is resistant to the one or more melanoma treatments or is at risk of developing resistance to the one or more melanoma treatments, by
i) measuring the activity and/or expression level of NAMPT in a biological sample obtained from the subject;
ii) comparing the activity and/or expression level measured at step i) with a corresponding predetermined reference value, and, when the activity and/or expression level of NAMPT is higher than the corresponding predetermined reference value, then performing the step of administering.

8. The method according to claim 6, wherein the inhibitor of NAMPT is a small organic molecule.

9. The method according to claim 6, wherein the inhibitor of NAMPT is FK866.

10. The method according to claim 6, wherein the inhibitor of NAMPT is CHS 828.

11. The method according to claim 6, wherein the inhibitor of NAMPT is a siRNA.

12. The method according to claim 6, wherein the inhibitor of NAMPT is a MEK inhibitor.

13. The method according to claim 6, wherein the inhibitor of NAMPT is ERK inhibitor.

14. A method of screening a drug suitable for the treatment of resistance to a treatment for melanoma comprising i) providing a test compound and ii) determining the ability of said test compound to inhibit the activity of NAMPT.
